(19) United States
(12) Patent Application Publication
(10) Pub. No.: US 2007/0286903 A1
(43) Pub. Date: Dec. 13, 2007

(54) Composition and method for taste masking

(76) Inventors: Brian T. Becicka, Charles City, IA (US); Erik T. Michalson, Charles City, IA (US)

Correspondence Address: BANNER & WITCOFF, LTD., 1100 13th STREET, N.W., SUITE 1200, WASHINGTON, DC, 20005-4051, US

(21) Appl. No.: 11/451,622

(22) Filed: Jun. 13, 2006

(51) Int. Cl. A61K 9/24

(52) U.S. Cl. 424/472

(57) ABSTRACT

The present invention relates to a taste-masked composition of an active pharmaceutical ingredient (API) for oral delivery and a related method for the preparation of the taste-masked composition comprising a granulated mixture of the active pharmaceutical ingredient, an insoluble matrix component, a film-forming agent, and a water soluble binder

### FIELD OF THE INVENTION

The present invention relates to a composition and method for the preparation of taste-masked active pharmaceutical ingredients (APIs) for oral delivery.

### BACKGROUND OF THE INVENTION

The preparation of a palatable dosage form of an active pharmaceutical ingredient (API) has long been an approach for helping to ensure patient compliance with a prescribed oral drug treatment regimen. Taste masking of an API, particularly those APIs which have an extremely unpleasant taste, has generally been attempted by some combination of (i) coating the API with a film, or (ii) forming a complex of the API with an ion-exchange matrix material.

For example, U.S. Pat. No. 5,075,114 describes a fluidized bed method of coating a pharmaceutical agent for taste-masking purposes. The patent describes the coating as a blend of cellulosic materials, i.e., hydroxypropyl cellulose and either cellulose acetate, cellulose acetate butyrate, or both. A number of drugs are mentioned, including ibuprofen, loperamide, famotidine, cimetidine, and ranitidine.

U.S. Pat. No. 5,082,669 describes ethyl cellulose coatings for bitter-tasting drugs. A number of drugs are mentioned as possibilities (at column 3, lines 13-31). The coating is prepared using either a film-forming solution or dispersion, or a spraying technique (column 5, lines 36-50).

Based on its Abstract, it appears that JP 57058631 describes coating a granulated API using a combination of an insoluble polymer coating agent, such as ethyl cellulose, and several polymer coating agents of varying solubility characteristics as a way of masking a bitter taste of a drug.

U.S. Pat. No. 5,032,393 suggests that the bitter taste of ranitidine can be masked by absorbing ranitidine hydrochloride onto a sulfonated styrene resin crosslinked with divinylbenzene or a methacrylic acid-divinylbenzene resin.

U.S. Pat. No. 3,594,470 and the related publication, Borodkin and Sundberg, J. of Pharmaceutical Sciences, 60(10):1523-1527 (1971), describe coating weak ion exchange resins previously complexed with basic-reacting APIs, such as dextromethorphan, with a mixture of ethylcellulose and hydroxypropylmethyl cellulose as a prelude to making chewable tablets.

Example XII of U.S. Pat. No. 4,851,226 describes a coating formulation for taste-masking loperamide, supplied as the HCl salt presumably in the form of granules having a particle size of 40-60 mesh, comprising a blend of cellulose acetate and polyvinylpyrrolidone.

In U.S. Pat. No. 5,075,114, loperamide, supplied as the HCl salt presumably in the form of granules having a particle size of 40-80 mesh, is taste-masked in Example X by a coating comprising a blend of cellulose acetate and hydroxypropyl cellulose.

U.S. Pat. No. 5,215,755 describes, in Example VIII, a coating formulation for taste-masking loperamide, supplied as the HCl salt presumably in the form of a powder having a particle size of 40-80 mesh, comprising a blend of hydroxyethyl cellulose and hydroxypropyl cellulose.

U.S. Pat. No. 5,489,436 describes a coating formulation for taste-masking loperamide in Example VIII comprising a mixture of cellulose acetate, polyvinylpyrrolidone and a copolymer of dimethylaminoethyl methacrylate and neutral methacrylic acid ester.

The art continues to explore new ways for taste masking APIs.

### DETAILED DESCRIPTION OF THE INVENTION

The present invention is directed to a composition and a related method for the preparation of taste-masked active pharmaceutical ingredients (APIs). Taste masking can be defined as the perceived reduction of an undesirable taste commonly associated with a particular API.

The present invention obtains the taste masking of an API by conventional granulating (e.g., rotogranulating) techniques. In accordance with the invention, the API is granulated using a combination of binders and a matrix component.

The present invention is suitable for taste-masking APIs that are insoluble in the granulating solvent. Applicants have observed that the success of the present composition for taste-masking an API is a function of the taste threshold of the API which in turn is a direct function of the solubility of the API. APIs which exhibit free solubility in the granulating solvent are not successfully taste-masked with the present invention. Thus, the API should exhibit only a slight solubility in the granulating solvent such that the process of the present invention provides an acceptable level of taste masking. Preferably, the API is only sparingly soluble in the granulating solvent, i.e., the API should preferably exhibit a level of solubility in the granulating solvent of less than 0.5 g per liter. For example, loperamide hydrochloride is considered to be only sparingly soluble in water and can be successfully taste-masked using the present invention when employing water as the granulating solvent.

Oftentimes the solubility of a particular API, which on first examination might not be considered applicable for taste-masking using the present invention, can be modified to a suitably low solubility in the granulating liquid of choice by using an alternative salt form of the API, by using an alternative crystal form of the API, by using the API in its free base or free acid form, or by pre-granulating the API to effectively render it non-soluble during the granulation procedure of the present invention.

In accordance with the present invention, a granular pharmaceutical preparation is produced by mixing an API having a low solubility in a granulating solvent (and preferably the API is sparingly soluble in the granulating solvent) with three essential ingredients: (A) an insoluble matrix component, (B) a film-forming agent, and (C) a water soluble binder and granulating the mixture. The various ingredients that are used are substantially pure and non-toxic. The granulated material so-produced then can be sized, and milled and made into tablets, capsules or a variety of other dosage forms as noted hereinafter.

One of the main advantages of the present invention is that by preparing the taste-masked API as a granular composition one is better able to obtain and/or control the particle size of the material destined for use in preparing the final dosage form. When using the prior art's approach of applying a taste-masking coating directly onto the API, it is very difficult to control particle size within specifically desired limits to the same degree. The prior art's alternative approach of forming a complex with an ion exchange agent is limited to APIs that have solubility and ionic characteristics suitable for that approach. Thus, the present invention permits taste masking of APIs that could not be accommodated with that technology.

The insoluble matrix component (A) of the composition of the present invention comprises one or more physiological inert particulate materials that are insoluble in water, including fluids of the digestive tract, and also are preferably insoluble in most of the organic solvents commonly used as granulating aides in the preparation of pharmaceutical products. Suitable insoluble matrix components include microcrystalline cellulose, powdered cellulose, cross-linked sodium carboxymethylcellulose (croscarmellose sodium), silica, clay, cross-linked polyvinylpyrrolidone (crospovidone), ion exchange resin materials (including cationic and anionic exchange resins themselves) and the like materials.

Ion exchange resin materials have proven to be suitable as the insoluble matrix component. Such resins include those made with styrene and cross-linked with divinylbenzene, cationic resins synthesized from dimethylaminoethyl methacrylate and methacrylic acid esters and cationic resins synthesized from (meth)acrylic acid crosslinked with divinylbenzene. The divinylbenzene-styrene resins are often provided as both cationic resins, for example having either sulfonate or carboxylate functional groups, and anionic resins, for example having amino groups. The preparation and commercial sources of such resins are well known to those versed in the art of taste-masking pharmaceuticals and require no further description.

In fact, since the present invention does not rely upon the ion exchange capacity of the resin, but only its ability to function as a matrix component of the granulated product, those resin materials that have not been functionalized with cationic and anionic exchange sites also should be suitable for use as the insoluble matrix component in this invention. Indeed, the phrase “ion exchange resin materials” is intended to embrace both the functionalized and non-functionalized resin materials. For example, non-functionalized divinylbenzene-styrene resins can be used as the insoluble matrix material.

The insoluble matrix component generally constitutes from 10 to 75 percent (%) by weight of an API-containing granule, preferably from 25 to 55 weight percent of the API-containing granule and usually from 35 to 45 weight percent of the API-containing granule.

The insoluble matrix component is supplied to the granulation process as a powder, i.e., in a particulate form, such that substantially all of the particles will be less than 500 microns (μm) in size, are usually less than 300 μm and preferably are less than 150 μm. The insoluble matrix component preferably has a particle size distribution in the range of 20 to 300 μm, and preferably has a number average particle size of less than 150 microns, for example a number average particle size in the range of 50 to 100 μm is generally preferred.

As used throughout this specification and claims, particle size is defined as the smallest pore size of a screen through which a particle will pass under a normal sieving operation, i.e., sieving without coincident milling. Generally, a particle size distribution can be assigned by identifying the screens through which substantially all of the particles will pass (e.g., over 98 weight percent of the particles will pass and though which substantially none of the particles will pass (e.g., less than 2 weight percent of the particles will pass.

The next constituent of the granular preparation is the film-forming agent (B). Component (B) can be selected from known pharmaceutical coating agents, many of which are polymeric materials and include cellulose-based coating agents; methacrylate-based coating agents, and polyvinyl acetate phthalate-based coating agents. Suitable cellulose-based coating agents include methyl, ethyl and propyl cellulose, cellulose acetate phthalate, hydroxypropyl methyl cellulose, hydroxypropylcellulose, hydroxypropyl ethyl cellulose, cellulose acetate, cellulose acetate butyrate, and nitrocellulose. Suitable methacrylate-based coating agents include anionic polymers of methacrylic acid and methacrylates with a COOH group, cationic polymers with a dimethylaminoethyl ammonium group, copolymers of acrylate and methacrylates with quaternary ammonium groups, copolymers of acrylate and methacrylates with quaternary ammonium group in combination with sodium carboxymethylcellulose, waxes and the like materials.

In many instances it will be preferred to use a film-forming agent that is insoluble in water but soluble in an organic solvent. Such a film forming agent would embrace the alkyl derivatives of cellulose, preferably cellulose ethers such as methyl, ethyl and propyl cellulose, which are insoluble in water and soluble in organic solvents; cellulose esters such as cellulose acetate and cellulose acetate butyrate; nitrocellulose; poly(meth)acrylates, polyvinyl acetate, polyvinyl chloride, waxes and the like. One preferred film-forming agent (B) comprises ethyl cellulose.

The film-forming agent can be supplied to the granulating process dissolved in an organic solvent, such as ethanol, which then can provide at least a portion of the granulating liquid. Preferably, however, the film-forming agent is provided as a water-based dispersion and comprises an aqueous dispersion of ethyl cellulose. A suitable ethyl cellulose aqueous dispersion will normally have a concentration of water-insoluble ethyl cellulose of 3 to 40 wt. %, more usually 10 to 35 wt. %. The ethyl cellulose aqueous dispersion is preferably used in combination with at least one physiologically compatible lipophilic diester of (i) a C₆-C₄₀ and preferably a C₁₀-C₁₆ aliphatic or aromatic dicarboxylic acid and (ii) a C₁-C₈ and preferably a C₂-C₅ aliphatic alcohol, as a plasticizer. Suitable plasticizers include dibutyl phthalate, diethyl phthalate, dibutyl sebacate and diethyl sebacate. Usually, the quantity of plasticizer is from 5 to 50 wt. % and preferably 10 to 40 wt. %, relative to ethyl cellulose.

The aqueous ethyl cellulose dispersion may be a commercial product such as, for example, sold under the names Aquacoat® or Surelease®. Such dispersions, such as for example Surelease®, may already contain the necessary plasticizer. Alternatively, it is possible to incorporate the plasticizers into the aqueous ethyl cellulose dispersion, possibly with the assistance of a surfactant or an emulsifier as needed.

The film-forming agent generally constitutes from 3 to 40 percent (%) by weight of the API-containing granule, preferably from 5 to 25 weight percent of the API-containing granule and more preferably from about 5 to 10 weight percent of the API-containing granule.

The other essential component in the granular pharmaceutical preparation of the present invention is the water soluble binder (C). Suitable ingredients for use as the water soluble binder include organic polyols (typically non-toxic hydrocarbons having two or more hydroxyls) such as 1,3-dihydroxypropane, hexylene glycol, glycerine, sorbitol, inositol and carbohydrates such as glucose and sucrose; polyethylene glycol; hydroxypropyl cellulose; hydroxypropyl methylcellulose; hydroxyethyl cellulose; polyvinyl alcohol; polyvinylpyrrolidone; carboxymethylcellulose and the like materials. Thus, a suitable water soluble binder material may be a PEG having a molecular weight of 500 or more (preferably between 1000 and 6000) or a polyvinylpyrrolidone having a molecular weight of at least 10000 (preferably between 10000 and 360,000). The preferred binder material is polyethylene glycol (PEG), particularly PEGs having a number average molecular weight of 3000 to 4000.

The water soluble binder generally constitutes from 3 to 40 percent (%) by weight of the API-containing granule, preferably from 10 to 30 weight percent of the API-containing granule and more preferably from about 15 to 25 weight percent.

In accordance with the present invention, the components (A) and (B) and the components (B) and (C) are normally provided in a relative amount ((A):(B)) of 1:1 to 12:1 and ((B):(C)) of 0.2:1 to 2:1, respectively. Preferably these components are provided in a relative amount of (A):(B) of 5:1 to 7:1 and of (B):(C) of 0.3:1 to 0.6:1.

The API is added to the other essential ingredients of the present composition in a desired quantity and in a suitable particle size. Preferably, the weight ratio of the API to the sum of ingredients (A), (B) and (C) is in the range of 0.1:1 to 3:1. Usually, the quantity of API will not exceed a weight ratio of about 1:1.

The API can be supplied to the granulation process as a pure medicament or can be pre-granulated with conventionally used binder materials and other common pharmaceutical excipients, including those substances identified above as suitable water soluble binders, before it is granulated with the three essential ingredients in accordance with this invention. Preferably, whether the API is provided as a pure medicament or in a pre-granulated form, the API is supplied to the granulation process in a particulate form, such that substantially all of the particles will be less than 300 microns (μm) in size. The solid API preferably has a particle size distribution in the range of 10 to 300 μm, for example a number average particle size in the range of approximately 50 to 150 μm and more preferably from 50 to 100 μm.

In preparing the granular preparation, a mixture of the solid ingredients may be wetted with a solvent in which one of components (B) and/or (C) is soluble in order to make the mixture suitable for a granulating operation. Alternatively, one of components (B) and (C) may be pre-dissolved directly in the granulating liquid.

Preferably, ethyl cellulose is used as the film-forming agent and PEG 3350 is used as the water soluble binder and the granulating liquid comprises a solution of the PEG 3350 in an aqueous dispersion of the ethyl cellulose. Suitable aqueous dispersions of ethyl cellulose were identified earlier. In any event, the taste-masked granular preparation of the present invention is preferably prepared by a wet-granulation procedure in which one of components (B) or (C), dissolved in a suitable solvent, is added with the API and along with component (A) into suitable granulation equipment and is blended therein to form granules. Following wet granulation, the granular material typically is dried and may be milled and sized to a desired particle size.

The resulting granules preferably have an average particle size of not more than about 150 microns, and more preferably the average particle size of the granules falls within the range of 50 to 100 microns, usually between 70 and 100 microns.

Thus, the wet granulation can be conducted using an organic solvent, or solvents, by using an aqueous solvent or by some combination of aqueous and organic solvents. Suitable organic solvents may include, but are not limited to the following: methanol, ethanol, isopropanol, acetone, chloroform, and methylene chloride. Preferably, the granulation is performed using an aqueous solvent in which component (C) is dissolved and component (B), as described above, is dispersed.

The resulting granular (e.g., granulated) material is suitable for making tablets, such as chewable tablets and orally disintegrating tablets, for making films, especially fast dissolving films, or for making capsules. Tablets can be further coated or glazed as desired to alter their release characteristics.

As alluded to earlier, in one variant of the above-described method, the API itself also can first be granulated using conventional techniques before it is added to the other essential ingredients of the granulation process of the present invention. Any known method of granulating the API can be utilized in this alternative embodiment.

Depending on the relative quantities of the various ingredients, (A), (B) and (C), both among themselves and relative to the API(s), in addition to taste masking, the practice of the present invention may also influence the release time the API(s) experiences as the medicament passes through a patient's digestive system. In other words, the taste-masked preparation of the present invention is capable of not only masking drugs with undesirable taste characteristics but may also control the rate at which the drug is delivered following oral administration to a patient. Thus, drugs whose target dissolution profile calls for immediate release can be efficiently taste masked using this invention, as can drugs that are preferably delivered using a desired time-release profile.

In the broad practice of the present invention, the API to be taste-masked may belong to any class of therapeutic agents which can be administered orally, i.e., by mouth. Thus, the designation “API” as used herein, is meant to include any therapeutic or otherwise active agent, preferably a pharmaceutical compound or chemical that is capable of being orally administered. The designation “API” is also intended to include nutritional supplements, particularly minerals and vitamins. Generally, the APIs used in conjunction with the present invention are those which are bitter or otherwise unpleasant-tasting and thus in need of taste masking.

The kinds of APIs that may benefit from the present invention include, without being limiting, antibiotics, antiviral agents, analgesics, anesthetics, anorexics, antiarthritics, antiasthmatic agents, anticonvulsants, antidepressants, antidiabetic agents, antidiarrheals, antihistamines, anti-inflammatory agents, antinauseants, antineoplastics, antiparkinsonism drugs, antipruritics, antipsychotics, antipyretics, antispasmodics, H₂ antagonists, antitussives, cardiovascular drugs, antiarrhythmics, antihypertensives, ACE inhibitors, diuretics, vasodilators, hormones, hypnotics, immunosuppressives, muscle relaxants, parasympatholytics, parasympathomimetics, psychostimulants, sedatives, antimigrane agents antituberculosis agents, tranquilizers vitamins and mineral supplements.

Mention may be made in particular of antibiotics such as tetracycline, penicillin V, or neomycin; hypnotics such as the barbiturates, methaqualone or mecloqualone; oral antidiabetics such as sulfamides or biguanides; antihistamines such as chlorpheniramine maleate, phenindamine tartrate, pyrilamine maleate, doxylamine succinate, phenyltoloxamine citrate, or promethazine; bronchodilators such as theophylline or hydroxyethyl theophylline; vasoconstrictors such as ephedrine or isoprenaline or naphazoline; and antitussants such as dextromethorphan, dextromethorphan hydrobromide, noscapine, carbetapentane citrate, and chlophedianol hydrochloride.

Other APIs for which the composition and process of the present invention are those which are preferably released in the gut or upper G-I tract and include but are not limited to acetaminophen, ibuprofen and its sodium salt, dexibuprofen lysinate, naproxen and its sodium salt, and other related NSAID's and their salts, gastrointestinal drugs (e.g. loperamide and famotidine) and decongestants (e.g. pseudoephedrine) as well as salts and combinations thereof.

As noted above, use of a particular API depends on the ability to prepare a form of the API having limited solubility, and preferably sparing solubility in the granulating solvent.

In most cases, standard granulating equipment and drying apparatus can be used to produce the present granular preparation and such equipment and apparatus are well known to those skilled in the art. For example, pan granulators and rotor granulators along with spray drying and drum drying procedures may be suitable. Preferred ways of performing the granulation include use of paddle dryers or fluidized bed plow mixers. As one suitable piece of equipment one can use the Tilt-A-Mix mixer available from Processall, Inc.

Thus, to prepare the granular composition of the present invention, the various ingredients typically are mixed in such granulating equipment in the presence of either an aqueous solution of the water soluble binder or an organic solution of the water-insoluble film-forming agent as the granulating liquid.

An advantage of the techniques used in practicing the present invention is that one can produce granules having a uniform distribution of the API. In this way, one can be confident that when these granules are used to prepare the ultimate oral dosage form, whether in the form of a film (such as a fast melt film), a tablet (including chewable tablets and fast dissolving tablets), a capsule, an oral suspension, a gum, a lozenge, or the like dosage forms, one is precisely providing the desired quantity of the API, and not an undesired lower or higher amount of the API.

When making a final dosage form using the API-containing granules of the present invention any of the wide variety of excipients commonly used in making pharmaceutical preparations can be used. For example, disintegrants, coloring agents, flavoring agents, lubricants, fillers and the like materials can be employed with the inventive granular composition of this invention. The present invention is not to be limited to any specific set of excipients.

As used in this specification and in the appended claims, the singular forms “a”, “an” and “the” also are intended to include plural referents unless the content clearly dictates otherwise. Thus, for example, reference to “an API” includes reference to one or more APIs (drugs), and the like.

The invention is further illustrated by the following example, which is not to be construed as limiting.

### EXAMPLE 1

Taste-masked Loperamide Hydrochloride: A premix of 3.4 g of dibutyl sebacate, 235 g water, 28.4 g of a 30% by weight dispersion of ethyl cellulose (Aqucoat® ECD) and 25 g of polyethylene glycol 3350 from Dow Chemical is prepared in a 1 liter round bottom flask with magnetic stirring. The mixture forms a milky suspension. Thereafter, 50 g of loperamide HCl and 50 g of a cationic resin (Amberlite® IRP64) are added to form a homogeneous mixture which is granulated using a Rotovap rotary evaporator under imposition of a vacuum and at a temperature 65° C. Any solid clumps that are formed are broken-up to produce a free-flowing solid that is dried to less than 5% moisture as shown by KF titration. The dried solid is then milled and sieved to a desired particle size to yield a granular product of the present invention.

The present invention has been described with reference to specific embodiments. However, this application is intended to cover those changes and substitutions that may be made by those skilled in the art without departing from the spirit and the scope of the invention. Unless otherwise specifically indicated, all percentages are by weight. Throughout the specification and in the claims the term “about” is intended to encompass + or −5% and preferably is only about + or −2%.

1. A taste-masked pharmaceutical preparation suitable for oral administration comprising a granulated mixture of an active pharmaceutical ingredient, an insoluble matrix component, a film-forming agent, and a water soluble binder.

2. The taste-masked pharmaceutical preparation of claim 1 wherein the insoluble matrix component is selected from the group consisting of an ion exchange resin material, microcrystalline cellulose, powdered cellulose, cross-linked sodium carboxymethylcellulose, silica, clay, cross-linked polyvinylpyrrolidone and mixtures thereof.

3. The taste-masked pharmaceutical preparation of claim 1 wherein the film-forming agent is selected from the group consisting of cellulose-based coating agents; methacrylate-based coating agents; polyvinyl acetate phthalate-based coating agents and mixtures thereof,

4. The taste-masked pharmaceutical preparation of claim 3 wherein the film-forming agent is selected from the group consisting of methyl cellulose, ethyl cellulose, propyl cellulose, cellulose acetate phthalate, hydroxypropyl methyl cellulose, hydroxypropylcellulose, hydroxypropyl ethyl cellulose, cellulose acetate, cellulose acetate butyrate, nitrocellulose, anionic polymers of methacrylic acid and methacrylates with a carboxyl group, cationic polymers with a dimethylaminoethyl ammonium group, copolymers of acrylate and methacrylates with quaternary ammonium groups, copolymers of acrylate and methacrylates with quaternary ammonium group in combination with sodium carboxymethylcellulose, waxes and mixtures thereof.

5. The taste-masked pharmaceutical preparation of claim 3 wherein the film-forming agent is selected from the group consisting of a cellulose ether, a cellulose ester, nitrocellulose, a poly(meth)acrylate, a polyvinyl acetate, a polyvinyl chloride, a wax and mixtures thereof.

6. The taste-masked pharmaceutical preparation of claim 1 wherein the water soluble binder is selected from the group consisting of an organic polyol; polyethylene glycol; hydroxypropyl cellulose; hydroxypropyl methylcellulose; hydroxyethyl cellulose; polyvinyl alcohol; polyvinylpyrrolidone; carboxymethylcellulose and mixtures thereof.

7. The taste-masked pharmaceutical preparation of claim 6 wherein the polyol is selected from 1,3-dihydroxypropane, hexylene glycol, glycerine, sorbitol, inositol, glucose, sucrose and mixtures thereof.

8. The taste-masked pharmaceutical preparation of claim 1 wherein the insoluble matrix component has a particle size of less than 300 microns.

9. The taste-masked pharmaceutical preparation of claim 1 wherein the insoluble matrix component has an average particle size of less than 150 microns.

10. The taste-masked pharmaceutical preparation of claim 1 wherein the active pharmaceutical ingredient has a particle size of less than 300 microns.

11. The taste-masked pharmaceutical preparation of claim 1 wherein the active pharmaceutical ingredient has an average particle size of less than 150 microns.

12. The taste-masked pharmaceutical preparation of claim 1 wherein the insoluble matrix component comprises from 10% to 75% by weight, the film-forming agent comprises from 3% to 40% by weight, and the water soluble binder comprises from 3% to 40% by weight of the taste-masked pharmaceutical preparation.

13. The taste-masked pharmaceutical preparation of claim 12 wherein the film-forming agent is an aqueous dispersion of ethyl cellulose, wherein the insoluble matrix component is an ion exchange resin material and wherein the water soluble binder is polyethylene glycol.

14. The taste-masked pharmaceutical preparation of claim 1 wherein the API is selected from antibiotics, antiviral agents, analgesics, anesthetics, anorexics, antiarthritics, antiasthmatic agents, anticonvulsants, antidepressants, antidiabetic agents, antidiarrheals, antihistamines, anti-inflammatory agents, antinauseants, antineoplastics, antiparkinsonism drugs, antipruritics, antipsychotics, antipyretics, antispasmodics, H₂ antagonists, antitussives, cardiovascular drugs, antiarrhythmics, antihypertensives, ACE inhibitors, diuretics, vasodilators, hormones, hypnotics, immunosuppressives, muscle relaxants, parasympatholytics, parasympathomimetics, psychostimulants, sedatives, antimigrane agents antituberculosis agents, tranquilizers vitamins and mineral supplements.

15. A method of making a taste-masked pharmaceutical preparation comprising granulating a mixture of an active pharmaceutical ingredient having a limited solubility in a granulating liquid, an insoluble matrix component, a film-forming agent and a water soluble binder with the granulating liquid.

16. The method of claim 15 wherein the insoluble matrix component is selected from the group consisting of an ion exchange resin material, microcrystalline cellulose, powdered cellulose, cross-linked sodium carboxymethylcellulose, silica, clay and cross-linked polyvinylpyrrolidone.

17. The method of claim 16 wherein the film-forming agent is selected from the group consisting of cellulose-based coating agents; methacrylate-based coating agents, polyvinyl acetate phthalate-based coating agents and mixtures thereof,

18. The method of claim 17 wherein the film-forming agent is selected from the group consisting of methyl cellulose, ethyl cellulose, propyl cellulose, cellulose acetate phthalate, hydroxypropyl methyl cellulose, hydroxypropylcellulose, hydroxypropyl ethyl cellulose, cellulose acetate, cellulose acetate butyrate, nitrocellulose, anionic polymers of methacrylic acid and methacrylates with a carboxyl group, cationic polymers with a dimethylaminoethyl ammonium group, copolymers of acrylate and methacrylates with quaternary ammonium groups, copolymers of acrylate and methacrylates with quaternary ammonium group in combination with sodium carboxymethylcellulose, waxes and mixtures thereof.

19. The method of claim 17 wherein the film-forming agent is selected from the group consisting of a cellulose ether, a cellulose ester, nitrocellulose, a poly(meth)acrylate, a polyvinyl acetate, a polyvinyl chloride, a wax and mixtures thereof

20. The method of claim 15 wherein the water soluble binder is selected from the group consisting of an organic polyol; polyethylene glycol; hydroxypropyl cellulose; hydroxypropyl methylcellulose; hydroxyethyl cellulose; polyvinyl alcohol; polyvinylpyrrolidone; carboxymethylcellulose and mixtures thereof.

21. The method of claim 20 wherein the polyol is selected from 1,3-dihydroxypropane, hexylene glycol, glycerine, sorbitol, inositol, glucose, sucrose and mixtures thereof.

22. The method of claim 15 wherein the wherein the insoluble matrix component is provided in an amount of from 10% to 75% by weight, the film-forming agent is provided in an amount of from 3% to 40% by weight, and the water soluble binder is provided in an amount of from 3% to 40% by weight of the taste-masked pharmaceutical preparation.
